(19) United States
(12) Patent Application Publication
(10) Pub. No.: US 2021/0371465 A1
(43) Pub. Date: Dec. 2, 2021

(54) METHOD OF USING A TLR9 ANTAGONIST AS AN ANTI-INFLAMMATORY AND ANTI-FIBROTIC AGENT

(71) Applicant: YALE UNIVERSITY, New Haven, CT (US)

(72) Inventor: Wajahat MEHAL, Branford, CT (US)

(21) Appl. No.: 17/213,299

(22) Filed: Mar. 26, 2021

Related U.S. Application Data
(60) Provisional application No. 62/994,923, filed on Mar. 26, 2020.
(60) Provisional application No. 63/105,428, filed on Oct. 26, 2020.

(51) Int. Cl. C07K 7/08; A61P 1/16

(52) U.S. Cl. CPC C07K 7/08; A61K 38/00; A61P 1/16

(57) ABSTRACT

Methods of treating or ameliorating inflammatory disorders and fibrosis are described. The methods include administering a therapeutically effective amount of an oligonucleotide inhibitor of TLR9 to an individual suffering from an inflammatory disorder or fibrosis. Also described are kits for treating or ameliorating inflammatory disorders and fibrosis.

### CROSS-REFERENCE TO RELATED APPLICATION

This application claims priority to U.S. Provisional Patent Application No. 62/994,923 entitled “METHOD OF USING A TLR9 ANTAGONIST AS AN ANTI-INFLAMMATORY AND ANTI-FIBROTIC AGENT,” filed Mar. 26, 2020, and U.S. Provisional Patent Application No. 63/105,428 entitled “METHOD OF USING A TLR9 ANTAGONIST AS AN ANTI-INFLAMMATORY AND ANTI-FIBROTIC AGENT” filed Oct. 26, 2020, the disclosures of all of which are incorporated herein by reference in their entireties.

### INCORPORATION-BY-REFERENCE OF MATERIAL SUBMITTED ON A COMPACT DISC OR AS A TEXT FILE VIA THE OFFICE ELECTRONIC FILING SYSTEM

This invention contains one or more sequences in a computer readable format in an accompanying text file titled “047162-7213US1_ST25.txt,” sized 4 Kbytes, the contents of which are incorporated herein by reference in their entirety.

### BACKGROUND

Inflammation in organs and tissues can result from a variety of environmental stressors. For example, steatohepatitis (inflammation of the liver tissues) can result from a variety of stressors such as excessive alcohol consumption, adverse drug reactions, and excessive calorie intake (obesity). Obesity-induced inflammation of the liver is the most commonly observed form of steatohepatitis, and is termed nonalcoholic steatohepatitis (NASH). If left untreated, individuals exhibiting NASH can progress to fibrosis and/or cirrhosis of the liver. Inflammation in other tissues, such as heart, lung, kidney, and the like, can also result in similarly serious inflammatory conditions if not adequately treated.

Members of the Toll-like receptor (TLR) family have been associated with inflammatory signaling and regulation in the body. TLR9 is an endosomal pattern recognition receptor for which CpG-rich bacterial DNA and mammalian self-DNA are ligands. The presence of TLR9 may be necessary for toxic liver inflammation and fibrosis, which has been demonstrated for 38212409.3 hepatic steatosis and inflammation in the choline-deficient amino acid-defined diet model of NASH. Although TLR9 is a desirable target for treating or ameliorating inflammation in the liver and other tissues, little is known about the identity of suitable TLR9 ligands or how the TLR9 pathway is activated in NASH or in other inflammatory disorders. Clinically useful TLR9 ligands could represent a significant advance in the treatment of inflammatory disorders and fibrosis.

There is consequently a need to develop methods capable of treating individuals suffering from inflammatory disorders and/or fibrosis. The present invention addresses this need.

### SUMMARY OF THE INVENTION

In various embodiments, a method of treating or ameliorating fibrosis, an inflammatory disorder, or a combination thereof is provided. The method includes administering a therapeutically effective amount of a composition comprising an oligonucleotide agent comprising the nucleotide sequence of SEQ ID NO: 1 to an individual having fibrosis, an inflammatory disorder, or a combination thereof. In certain embodiments, the oligonucleotide agent consists essentially of the nucleotide sequence of SEQ ID NO: 1. In other embodiments, the oligonucleotide agent consists of the nucleotide sequence of SEQ ID NO: 1.

In various embodiments, a method of treating or ameliorating fibrosis, an inflammatory disorder, or a combination thereof is provided. The method includes administering a therapeutically effective amount of a composition comprising an oligonucleotide agent comprising the nucleotide sequence of SEQ ID NO: 1 to an individual having fibrosis, an inflammatory disorder, or a combination thereof. In certain embodiments, the therapeutically effective amount comprises about 0.05 mg/kg to about 2 mg/kg of the agent. In other embodiments, the method substantially reduces cellular or plasma levels of at least one pro-inflammatory cytokine chosen from IL-1β, IL-6, IL-8, IL-18, TNFα, or any combinations thereof, in the individual. In yet other embodiments, at least a portion of the cellular or plasma levels of the at least one pro-inflammatory cytokine results from activation of TLR9. In yet other embodiments, the oligonucleotide agent consists essentially of the nucleotide sequence of SEQ ID NO: 1. In yet other embodiments, the oligonucleotide agent consists of the nucleotide sequence of SEQ ID NO: 1.

In various embodiments, a kit that includes a composition having an oligonucleotide agent comprising the nucleotide sequence of SEQ ID NO: 1, an applicator, and instructional material for use thereof is provided. The instructional material includes instructions for treating or ameliorating fibrosis, an inflammatory disorder, or a combination thereof, in an individual. In certain embodiments, the oligonucleotide agent consists essentially of the nucleotide sequence of SEQ ID NO: 1. In other embodiments, the oligonucleotide agent consists of the nucleotide sequence of SEQ ID NO: 1.

### BRIEF DESCRIPTION OF THE FIGURES

The drawings illustrate generally, by way of example, but not by way of limitation, various embodiments of the present application.

FIG. 1A shows a liver section from the liver histology of mice after 26 weeks on regular chow diet, when mice were 32 weeks of age. The liver section is shown at 4× optical magnification.

FIG. 1B shows a liver section from the liver histology of mice after 26 weeks on regular chow diet, when mice were 32 weeks of age. The liver section is shown at 20× optical magnification.

FIG. 1C shows a liver section from the liver histology of mice after 33 weeks on regular chow diet, when mice were 39 weeks of age. The liver section is shown at 4× optical magnification.

FIG. 1D shows a liver section from the liver histology of mice after 33 weeks on regular chow diet, when mice were 39 weeks of age. The liver section is shown at 20× optical magnification. With increasing age, mild steatosis develops even on regular chow.

FIG. 2A shows a liver section from the liver histology of mice after 26 weeks on high fat diet (HFD), already showing the development of steatosis. The liver section is shown at 4× optical magnification.

FIG. 2B shows a liver section from the liver histology of mice after 26 weeks on high fat diet. The liver section is shown at 20× optical magnification.

FIG. 2C shows a liver section from the liver histology of mice after 33 weeks on high fat diet. The liver section is shown at 4× optical magnification. The mice exhibit liver steatosis.

FIG. 2D shows a liver section from the liver histology of mice after 33 weeks on high fat diet. The liver section is shown at 20× optical magnification.

FIG. 2E shows a liver section from the liver histology of mice after 33 weeks on high fat diet after treatment with OCR7314 (SEQ ID NO: 1) for 6 weeks, starting at week 27. The liver section is shown at 4× optical magnification.

FIG. 2F shows a liver section from the liver histology of mice after 33 weeks on high fat diet after treatment with OCR7314 (SEQ ID NO: 1) for 6 weeks, starting at week 26. The liver section is shown at 20× optical magnification.

FIG. 3A shows the steatosis grade assessment for wildtype mice fed with HFD (Research Diets Inc, #D12451), or regular chow (Harlan Teklad #TD.2916) starting at age 6 weeks. After 26 weeks of HFD the presence of NASH (non-alcoholic steatohepatitis) was confirmed by histology. At 27 weeks the HFD mice were divided into 2 groups (n=5 each) that were given the oligonucleotide OCR7314 (SEQ ID NO:1) (1 mg/kg/week, sc) for 6 weeks or control. Histological grading for steatosis, inflammation and fibrosis was conducted after blinding to the identity of the sample at low power (×4 magnification) with a grading in whole digits between 0 and 4. For each mouse liver six regions were randomly selected and graded. The data were statistically analyzed by One way ANOVA with Tukey's multiple comparison test. The mean difference of P≤0.05 was considered as significant. All the data were expressed as Mean±S.D. The statistical analysis were performed using Graphpad Prism 8.0 software.

FIG. 3B shows the inflammation grade assessment for wildtype mice fed with HFD (Research Diets Inc, #D12451), or regular chow (Harlan Teklad #TD.2916) starting at age 6 weeks. After 26 weeks of HFD the presence of NASH (non-alcoholic steatohepatitis) was confirmed by histology. At 27 weeks the HFD mice were divided into 2 groups (n=5 each) that were given the oligonucleotide OCR7314 (SEQ ID NO:1) (1 mg/kg/week, sc) for 6 weeks or control.

FIG. 3C shows the fibrosis grade assessment for wildtype mice fed with HFD (Research Diets Inc, #D12451), or regular chow (Harlan Teklad #TD.2916) starting at age 6 weeks. After 26 weeks of HFD the presence of NASH (non-alcoholic steatohepatitis) was confirmed by histology. At 27 weeks the HFD mice were divided into 2 groups (n=5 each) that were given the oligonucleotide OCR7314 (SEQ ID NO:1) (1 mg/kg/week, sc) for 6 weeks or control.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Definitions

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “fibrosis” as used herein refers to the formation of excessive connective tissue in an organ or other tissue. Fibrosis can interfere with the normal functioning of organs or tissues, and organs and tissues exhibiting fibrosis can be abnormally thickened or scarred.

The term “inflammatory disorder” as used herein refers to disorders such as non-alcoholic steatohepatitis (NASH), alcoholic hepatitis, pancreatitis, uveitis, encephalitis, nephritis, and/or crush injury. Inflammatory disorders can be generally characterized as exhibiting one or more of redness, pain, swelling, heat, or loss of function (partial or complete) in the affected organ or tissue.

The term “crush injury” as used herein refers to an injury to one or more tissues that results from compression of the tissue by an external force. These tissue injuries provoke an inflammatory response in the body at and/or near the site of injury.

The term “oligonucleotide” as used herein can include single- and double-stranded DNA (ssDNA and dsDNA), single- and double-stranded RNA (ssRNA and dsRNA), chemically modified polynucleotides or polynucleosides, and combinations thereof. Polynucleotides are polymers of nucleotides joined through phosphodiester or phosphorothioate linkages. A nucleoside consists of an adenine (A), guanine (G), cytosine (C), thymine (T), or uracil (U) bonded to a sugar moiety. A nucleotide is a phosphate ester of a nucleoside. The nucleoside units in DNA contain deoxyribose sugars.

The term “3′” refers to a region or position in a single-stranded polynucleotide or oligonucleotide that is downstream from another region or position in the same polynucleotide or oligonucleotide. The term “3′ end” refers to the 3′ terminus of the polynucleotide containing a 3′ phosphate group.

The term “5′” refers to a region or position in a polynucleotide or oligonucleotide upstream from another region or position in the same polynucleotide or oligonucleotide. The term “5′ end” refers to the 5′ terminus of the polynucleotide containing a 5′ phosphate group.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “individual” as used herein refers to any animal, or cells thereof whether in vitro or in situ, amenable to the methods described herein. In a non-limiting embodiment, the individual is a human.

The unit “mg/kg” as used herein refers to an amount of the therapeutic agent (in milligrams) per weight of treated individual (in kilograms). A dose of 1 mg/kg in an individual that weighs 70 kg would be equal to a dose of 70 mg of the therapeutic agent.

The term “instructional material,” as used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

A “therapeutic” treatment is a treatment administered to a subject who exhibits signs of pathology, for the purpose of diminishing or eliminating those signs.

The term “substantially reduce” as used herein refers to reducing the amount of a majority of, or mostly, a substance, by at least about 5%, 10%, 15%, 20%, 30%, 40%, 50%, 60%, 70%, 80%, 90%, 95%, 96%, 97%, 98%, 99%, 99.5%, 99.9%, 99.99%, or at least about 99.999% or more, or 100%, relative to an initial amount of the substance.

The term “equivalent dose” as used herein refers to a dose of a substance that is the same mass of substance per unit weight of an individual as another reference substance. For example, if the a therapeutic dose of substance A is 0.5 mg/kg, an equivalent dose of substance B would also be 0.5 mg/kg, even if the respective molecular weights of substance A and B are different.

The phrase “mRNA corresponding to at least one pro-inflammatory and/or pro-fibrotic cytokine” as used herein means an mRNA that can become translated by cellular machinery, such as by a ribosome, into a peptide that can ultimately act as a pro-inflammatory and/or pro-fibrotic cytokine.

### Methods of Using the Oligonucleotide Therapeutic Agent

A method of treating or ameliorating fibrosis, an inflammatory disorder, or a combination thereof is provided. The method includes administering a therapeutically effective amount of a composition comprising an oligonucleotide agent comprising the nucleotide sequence of SEQ ID NO: 1 to an individual having fibrosis, an inflammatory disorder, or a combination thereof. The method can treat the inflammatory disorder and prevent the inflammatory disorder from progressing to fibrosis. Treating an individual having an inflammatory disorder to prevent progression to fibrosis can include administering the oligonucleotide agent comprising the nucleotide sequence of SEQ ID NO: 1 to substantially reduce the plasma and cellular levels of at least one pro-inflammatory and/or pro-fibrotic cytokine chosen from IL-1β (interleukin-1β), IL-6, IL-8, IL-18, TNFα, IL-4, IL-5, IL-13, IL-17, type 1 interferon, TGF-β, or combinations thereof. Treating an individual having an inflammatory disorder to prevent progression to fibrosis can include administering the oligonucleotide agent comprising the nucleotide sequence of SEQ ID NO: 1 to substantially reduce tissue mRNA levels of mRNA corresponding to at least one pro-inflammatory and/or pro-fibrotic cytokine chosen from IL-1β (interleukin-1β), IL-6, IL-8, IL-18, TNFα (Tissue Necrosis Factor α), IL-4, IL-5, IL-13, IL-17, type 1 interferon, TGF-β, or combinations thereof.

Cytokine levels can be measured using recognized techniques in the art. For example, cytokine levels can be measured by measuring mRNA transcript levels, by using enzyme-linked immunosorbent assays (ELISA), by using surface-modified polystyrene beads (e.g., LUMINEX® 100 or 200 system, BD™ Cytokine Bead Array, and the like) or by using solid-state microchip arrays (e.g., Invitrogen™ Cytokine 30-Plex Human Panel, and the like).

mRNA levels can be measured using recognized techniques in the art. For example, mRNA levels can be measured using Northern blots (e.g., NorthernMax™ Kits from ThermoFisher Scientific), nuclease protection assays (e.g., RPA III™ Kit from ThermoFisher Scientific), or RT-PCR for quantitative determination of mRNA levels (e.g. RETROscript™ Kit and/or QuantumRNA™ Kits from ThermoFisher Scientific).

In various embodiments, the oligonucleotide agent comprising the following nucleotide sequence: 5′-TGC TCC TGG AGX GGT TGT-3′ (SEQ ID NO: 1), where X is deoxy-inosine. Oligonucleotides such as SEQ ID NO: 1 can be prepared according to previously described methods, such as in U.S. Pat. Nos. 8,759,305; 8,962,579; 9,476,053; 8,940,310; and 9,347,064, each of which is hereby incorporated by reference in its entirety. Activation of TLR9 can result in the production of pro-inflammatory cytokines mediated by NF-κB as well as by activation of IRF7 (Interferon Regulatory Factor 7). The oligonucleotide agent of SEQ ID NO: 1 can bind to TLR9 and reduce the inflammatory effects caused or mediated by its activation. The method can, in various embodiments, substantially reduce the cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations of these cytokines, where at least a portion of the cellular or plasma levels of the at least one-pro inflammatory cytokine results from activation of TLR9. The method can, in various embodiments, substantially reduce tissue mRNA levels of mRNA corresponding to at least one pro-inflammatory cytokine chosen from IL-1β, IL-6, IL-8, IL-18, TNFα, IL-4, IL-5, IL-13, IL-17, type 1 interferon, TGF-β, or combinations of these cytokines, where at least a portion of the tissue mRNA levels of the at least one-pro inflammatory cytokine results from activation of TLR9. Additionally, the method can reduce the transcriptional activity of IRF7 in an individual suffering from an inflammatory disorder and/or fibrosis.

In various embodiments, the method can substantially reduce the cellular or plasma levels of at least one pro-inflammatory cytokine chosen from IL-1β, IL-6, IL-8, IL-18, TNFα, IL-4, IL-5, IL-13, IL-17, type 1 interferon, TGF-β, to a level that is lower than the level that can be achieved with an equivalent dose of an oligonucleotide agent having the following nucleotide sequence: 5′-TGC TCC TGG AGG GGT TGT-3′ (SEQ ID NO: 2). In various embodiments, the method can substantially reduce tissue mRNA levels of mRNA corresponding to at least one pro-inflammatory cytokine chosen from IL-1β, IL-6, IL-8, IL-18, TNFα, IL-4, IL-5, IL-13, IL-17, type 1 interferon, TGF-β, to a level that is lower than the level that can be achieved with an equivalent dose of an oligonucleotide agent having the following nucleotide sequence of SEQ ID NO: 2. The method can also substantially reduce the transcriptional activity of IRF7 in an individual suffering from an inflammatory disorder and/or fibrosis to a level that is lower than the level that can be achieved with an equivalent dose of an oligonucleotide agent having the sequence SEQ ID NO: 2. Without being bound by theory, the oligonucleotide of SEQ ID NO: 1 can reduce pro-inflammatory and/or pro-fibrotic cytokine levels more effectively than the oligonucleotide of SEQ ID NO: 2 at least in part due to the presence of deoxy-inosine in the oligonucleotide of SEQ ID NO: 1, which allows for increased stability and in vivo activity.

TLR9 can also be activated by mitochondrial DNA (mtDNA) or nuclear DNA (nDNA), which can result in the production of any of the pro-inflammatory cytokines described herein. Mitochondrial DNA can be present as free mtDNA in solution, as intact extracellular mitochondria, or as mitochondria encapsulated in extracellular microparticles (MPs). In certain embodiments, tissue stress and damage in an individual can result in elevated levels of mtDNA or nDNA in the plasma of the individual. Tissue damage can result in the release of mtDNA or nDNA and ultimately lead to inflammation and/or fibrosis of the damaged tissue. For example, the level of mtDNA in the form of hepatocyte-originating MPs is significantly elevated in individuals with NASH. The presence of such MPs can be detected in plasma by, for example, flow cytometry after staining with MitoTracker Deep Red, which stains mitochondria with an intact membrane potential. These MPs can be identified by, for example, a combination of size gating (0.2-1.0 μm) and positivity for the plasma membrane dye PKH67 green. Dual staining with MitoTracker Deep Red and PKH67 allows for the identification of mitochondria enclosed within a plasma membrane. Other suitable techniques for identifying and staining mitochondria can also be used.

In various embodiments, the method substantially reduces cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual, wherein at least a portion of the cellular or plasma levels of the at least one pro-inflammatory cytokine result from activation of TLR9 by mtDNA or nDNA. In various embodiments, the method substantially reduces tissue mRNA levels of mRNA corresponding to at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual, wherein at least a portion of the tissue mRNA level corresponding to at least one pro-inflammatory cytokine results from activation of TLR9 by mtDNA or nDNA.

The method can, in various embodiments, reduce the cellular or plasma levels of pro-inflammatory cytokines chosen from IL-1β, IL-6, IL-8, IL-18, TNFα, IL-4, IL-5, IL-13, IL-17, type 1 interferon, TGF-β, or combinations thereof, that result from activation of TLR9 by mtDNA or nDNA to a level that is lower than the level that can be achieved with an equivalent dose of an oligonucleotide agent having the formula SEQ ID NO: 2. The method can, in various embodiments, reduce the tissue mRNA levels of mRNA corresponding to pro-inflammatory cytokines chosen from IL-1β, IL-6, IL-8, IL-18, TNFα, IL-4, IL-5, IL-13, IL-17, type 1 interferon, TGF-β, or combinations thereof, that result from activation of TLR9 by mtDNA or nDNA to a level that is lower than the level that can be achieved with an equivalent dose of an oligonucleotide agent having the formula SEQ ID NO: 2.

In various embodiments, the method can treat fibrosis of lung tissue, liver tissue, heart tissue, skin, lymphatic tissue, pancreatic tissue, intestinal tissue, kidney tissue, bone marrow, and/or connective tissue. Connective tissue can include cartilage, tendons, ligaments, or combinations of these tissues. The method can also treat inflammation or fibrosis in tissues that are not normally part of an organ system, such as inflammation or fibrosis in post-surgical scars. The inflammatory disorder can include, in various embodiments, non-alcoholic steatohepatitis (NASH), alcoholic hepatitis, pancreatitis, uveitis, encephalitis, nephritis, bronchitis, pneumonitis, pleuritic, chronic pulmonary obstructive disease (COPD), and/or crush injury. In various embodiments, the inflammatory disorder is NASH.

The method can, in various embodiments, lead to improvement in clinical markers related to NASH such as those measured by MRI-PDFF (MRI Proton Density Fat Fraction) and by Fibroscan ultrasound machines.

### Routes of Administration

In various embodiments, the compositions described herein can be administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration. The composition, in various embodiments, can be administered to the individual by subcutaneously.

For parenteral administration, the compositions described herein can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulating agents such as suspending, stabilizing and/or dispersing agents can be used.

The compositions described herein suitable for parenteral administration can be formulated in USP water, water for injection, sterile isotonic solutions such as saline and phosphate buffered saline for injection, and can also include pH buffers, salts, bulking agents, preservatives, and other pharmaceutically acceptable excipients.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use can also be presented as hard gelatin capsules wherein the active ingredient is mixed with an inert diluent.

Additional dosage forms suitable for use with the compositions described herein can include dosage forms as described in U.S. Pat. Nos. 6,340,475; 6,488,962; 6,451,808; 5,972,389; 5,582,837; and 5,007,790. Additional dosage forms suitable for use with the compositions described herein can include dosage forms as described in U.S. Patent Applications Nos. 20030147952; 20030104062; 20030104053; 20030044466; 20030039688; and 20020051820. Additional dosage forms suitable for use with the compositions described herein can include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Dosing and Dosage Regimens

The regimen of administration can affect what constitutes an effective amount. The therapeutic formulations can be administered to the subject either prior to or after the onset of a disease or disorder contemplated in the invention. Further, several divided dosages, as well as staggered dosages can be administered daily, weekly, or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present invention to a patient, preferably a mammal, more preferably a human, can be carried out using known procedures, at dosages and for periods of time effective to treat a disease or disorder contemplated in the invention. An effective amount of the therapeutic compound necessary to achieve a therapeutic effect can vary according to factors such as the state of the disease or disorder in the individual; the age, sex, and weight of the patient; and the ability of the therapeutic composition to treat a disease or disorder described herein. Dosage regimens can be adjusted to provide the optimum therapeutic response. For example, several divided doses can be administered daily or the dose can be proportionally reduced as indicated by the exigencies of the therapeutic situation. A person of ordinary skill in the art would be able to study the relevant factors and make the determination regarding the effective amount of the therapeutic compound without undue experimentation.

Actual dosage levels of the active ingredients in the pharmaceutical compositions described herein can be varied so as to obtain an amount of the active ingredient that is effective to achieve the desired therapeutic response for a particular patient, composition, and mode of administration, without being toxic to the individual.

A medical doctor, e.g., physician or veterinarian, having ordinary skill in the art can readily determine and prescribe or administer the effective amount of the pharmaceutical composition required. For example, the physician or veterinarian could start doses of the compositions described at levels lower than that required in order to achieve the desired therapeutic effect and gradually increase the dosage until the desired effect is achieved.

The frequency of administration of the compositions described herein varies from individual to individual depending on many factors including, but not limited to, age, disease or disorder to be treated, gender, overall health, and other factors. Thus, the methods described herein should not be construed to be limited to any particular dosage regimen and the precise dosage and composition to be administered to any individual is determined by the attending physician taking all other factors about the individual into account.

In various embodiments, the therapeutically effective amount can include about 0.01 mg/kg to about 5 mg/kg of the oligonucleotide agent. The therapeutically effective amount can include about 0.1 mg/kg to about 5 mg/kg, about 0.2 mg/kg to about 5 mg/kg, about 0.4 mg/kg to about 5 mg/kg, about 0.6 mg/kg to about 5 mg/kg, about 0.8 mg/kg to about 5 mg/kg, about 1 mg/kg to about 5 mg/kg, about 1.5 mg/kg to about 5 mg/kg, about 2 mg/kg to about 5 mg/kg, about 2.5 mg/kg to about 5 mg/kg, about 3 mg/kg to about 5 mg/kg, about 3.5 mg/kg to about 5 mg/kg, about 4 mg/kg to about 5 mg/kg of the agent.

In various embodiments, when the therapeutically effective amount of the composition is administered, the therapeutically effective amount can include about 0.05 mg/kg to about 2 mg/kg of the agent. The therapeutically effective amount can include about 0.1 mg/kg to about 2 mg/kg, about 0.2 mg/kg to about 2 mg/kg, about 0.3 mg/kg to about 2 mg/kg, about 0.4 mg/kg to about 2 mg/kg, about 0.5 mg/kg to about 2 mg/kg, about 0.5 mg/kg to about 1.8 mg/kg, about 0.5 mg/kg to about 1.6 mg/kg, about 0.5 mg/kg to about 1.4 mg/kg, about 0.5 mg/kg to about 1.2 mg/kg of the agent. The therapeutically effective amount can include about 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.2 mg/kg, 1.4 mg/kg, 1.6 mg/kg, 1.8 mg/kg, 2 mg/kg, 2.2 mg/kg, 2.4 mg/kg, 2.6 mg/kg, 2.8 mg/kg, 3 mg/kg, 3.2 mg/kg, 3.4 mg/kg, 3.6 mg/kg, 3.8 mg/kg, 4 mg/kg, 4.2 mg/kg, 4.4 mg/kg, 4.6 mg/kg, 4.8 mg/kg, or 5 mg/kg of the agent.

The method can include, in various embodiments, administering 0.01 mg/kg/week to about 5 mg/kg/week of the composition for about 1 week to about 12 weeks, or more. Treatment of an individual can continue indefinitely, including for the life-time of an individual. In various embodiments, the composition can be administered for about 1 week, 2 weeks, 3 weeks, 4 weeks, 5 weeks, 6 weeks, 7 weeks, 8 weeks, 9 weeks, 10 weeks, 11 weeks, or 12 weeks. There is no particular limit as to how long the composition described herein can be administered. The length of time for which the composition is administered can depend on the nature and severity of the inflammatory and/or fibroid disorder the individual suffers from. In situations where chronic administration is necessary to treat the individual, the compositions described herein can be administered chronically. In various embodiments, the composition can be administered for about 3, 4, 5, 6, 7, 8, 9, 10, 11, 12, 13, 14, 15, 16, 17, 18, 19, 20, 21, 22, 23, or 24 months, or more. The treatment can, in various embodiments, can contain a pause whereby no composition is administered to the individual for a period, followed by, resumption of administration of the composition. The pause period can be any suitable length of time that does not substantially decrease the effectiveness of the treatments described herein.

In various embodiments, the method includes administering about 0.1 mg/kg/week to about 1 mg/kg/week, about 0.2 mg/kg/week to about 1 mg/kg/week, about 0.3 mg/kg/week to about 1 mg/kg/week, about 0.4 mg/kg/week to about 1 mg/kg/week, or about 0.5 mg/kg/week to about 1 mg/kg/week of the composition for about 1 week to about 12 weeks, or more, or more. The method can include administering about 0.05 mg/kg/week, 0.1 mg/kg/week, 0.2 mg/kg/week, 0.3 mg/kg/week, 0.4 mg/kg/week, 0.5 mg/kg/week, 0.6 mg/kg/week, 0.7 mg/kg/week, 0.8 mg/kg/week, 0.9 mg/kg/week, or 1 mg/kg/week of the composition.

In various embodiments, the method includes administering 0.01 mg/kg/week to about 5 mg/kg/week of the composition for a first period at a first frequency and for a second period at a second frequency, wherein the second frequency is lower than the first frequency. In various embodiments, the method can include administering about 0.1 mg/kg/week to about 1 mg/kg/week, about 0.2 mg/kg/week to about 1 mg/kg/week, about 0.3 mg/kg/week to about 1 mg/kg/week, about 0.4 mg/kg/week to about 1 mg/kg/week, or about 0.5 mg/kg/week to about 1 mg/kg/week of the composition for a first period at a first frequency and for a second period at a second frequency, wherein the second frequency is lower than the first frequency. In various embodiments, the method can include administering about 0.05 mg/kg/week, 0.1 mg/kg/week, 0.2 mg/kg/week, 0.3 mg/kg/week, 0.4 mg/kg/week, 0.5 mg/kg/week, 0.6 mg/kg/week, 0.7 mg/kg/week, 0.8 mg/kg/week, 0.9 mg/kg/week, or 1 mg/kg/week of the composition for a first period at a first frequency and for a second period at a second frequency, wherein the second frequency is lower than the first frequency.

When the second frequency is lower than the first frequency, the method of administration described herein results in front loading of the oligonucleotide agent in the individual, followed by administering the composition containing the oligonucleotide agent less frequently. Such a dosing regimen can be advantageous to the individual due to decreased amounts of therapeutic agent used and by increased compliance due to the decreased frequency of dosing after the initial front loading period.

In various embodiments, the first period is about 2 weeks to about 6 weeks, the first frequency is weekly, the second period is about 2 weeks to about 10 weeks, and the second frequency is about every 2 weeks to about every 6 weeks.

In various embodiments, the composition includes at least one additional therapeutic agent. Non-limiting examples of suitable additional therapeutic agents include analgesics, non-steroidal anti-inflammatories (NSAIDs), corticosteroids, antibiotics, antivirals, immunosuppressants, metabolic pathway inhibitors, nuclear receptor agonists, hormone receptor agonists, kinase inhibitors, and the like.

In various embodiments, a method of treating or ameliorating fibrosis, an inflammatory disorder, or a combination thereof is provided. The method includes administering a therapeutically effective amount of a composition including an oligonucleotide agent comprising the nucleotide sequence of SEQ ID NO: 1 to an individual having fibrosis, an inflammatory disorder, or a combination thereof, wherein the therapeutically effective amount comprises about 0.05 mg/kg to about 2 mg/kg of the agent; wherein the method substantially reduces cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual; and wherein at least a portion of the cellular or plasma levels of the at least one pro-inflammatory cytokine result from activation of TLR9.

In various embodiments, the compositions described herein are formulated using one or more pharmaceutically acceptable excipients or carriers. In various embodiments, the pharmaceutical compositions of the invention can include a therapeutically effective amount of a compound comprising the nucleotide sequence of SEQ ID NO: 1 and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present invention are not limited to the particular formulations and compositions that are described herein.

### Kits

In various embodiments, a kit that includes a composition having an oligonucleotide agent comprising the nucleotide sequence of SEQ ID NO: 1, an applicator, and instructional material for use thereof is provided. The instructional material includes instructions for treating or ameliorating fibrosis, an inflammatory disorder, or a combination thereof, in an individual. The instructional material can include instructions for administering 0.01 mg/kg/week to about 5 mg/kg/week of the agent to the individual for about 1 week to about 12 weeks. The instructional material can include instructions for administering 0.01 mg/kg/week to about 5 mg/kg/week of the agent to the individual for a first period at a first frequency and for a second period at a second frequency, wherein the second frequency is lower than the first frequency. The instructional material can also include instructions for treating or ameliorating the inflammatory disorder to prevent the inflammatory disorder from progressing to fibrosis.

In various embodiments, the kit contains instructions for treating or ameliorating fibrosis. In other embodiments, the fibrosis includes fibrosis of lung tissue, liver tissue, heart tissue, skin, lymphatic tissue, pancreatic tissue, intestinal tissue, kidney tissue, bone marrow, and/or connective tissue.

In various embodiments, the kit contains instructions for treating or ameliorating an inflammatory disorder. In other embodiments, the inflammatory disorder includes non-alcoholic steatohepatitis (NASH), alcoholic hepatitis, pancreatitis, uveitis, encephalitis, nephritis, and/or crush injury.

In various embodiments, the kit includes an applicator that is suitable for subcutaneous administration of the compositions described herein. In various embodiments, the composition includes at least one additional therapeutic agent. In various embodiments, the kit includes a composition that contains at least one pharmaceutically acceptable excipient.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The examples described herein illustrate aspects of the present disclosure. However, they are in no way a limitation of the teachings or disclosure of the present disclosure as set forth herein.

The examples described herein are provided for purposes of illustration only, and are not intended to be limiting unless otherwise specified. Thus, the disclosure should in no way be construed as being limited to the examples described herein, but rather, should be construed to encompass any and all variations which become evident as a result of the teaching provided herein.

Without further description, it is believed that one of ordinary skill in the art can, using the description and illustrative examples, make and utilize the compounds of the present disclosure and practice the claimed methods. The working examples therefore, specifically point out selected embodiments of the present disclosure, and are not to be construed as limiting in any way the remainder of the disclosure.

The disclosures of each and every patent, patent application, and publication cited herein are hereby incorporated herein by reference in their entirety.

While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Examples

C57BL/6 mice were fed with high fat diet (HFD) (Research Diets Inc, #D12451, 45% calories from fat, 35% from carbohydrate, 20% from protein), or chow (Harlan Teklad #TD.2916 12% calories from fat, 48.5% from carbohydrate, 16.4% from protein) starting at age 6 weeks. At 26 weeks of HFD the presence of NASH was confirmed by histology and they were divided into two groups (n=5) at week 27 of HFD. One group of mice continued on the HFD and were administered the TLR9 antagonist of SEQ ID NO: 1 for 6 weeks. The second group of mice continued on the HFD and control for 6 weeks. Mice were sacrificed after 33 weeks of HFD (39 weeks of age) and liver tissue examined.

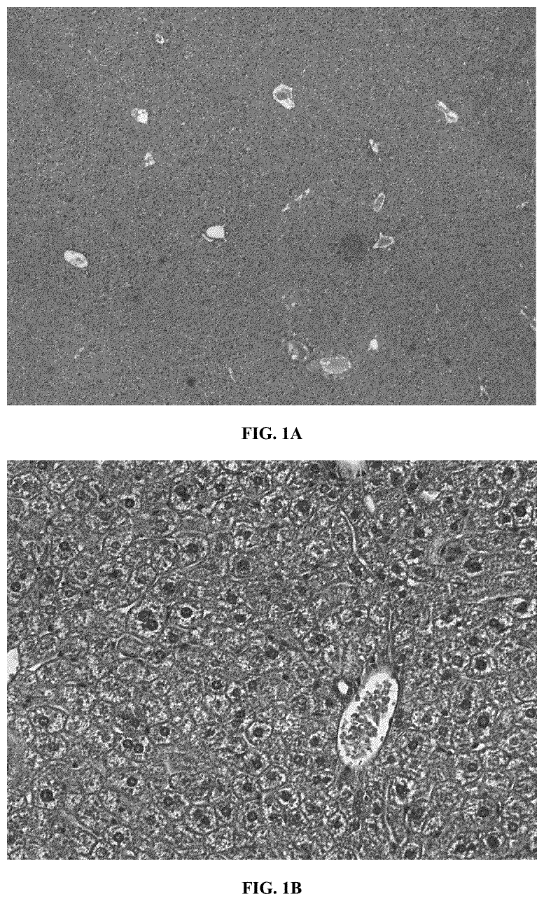

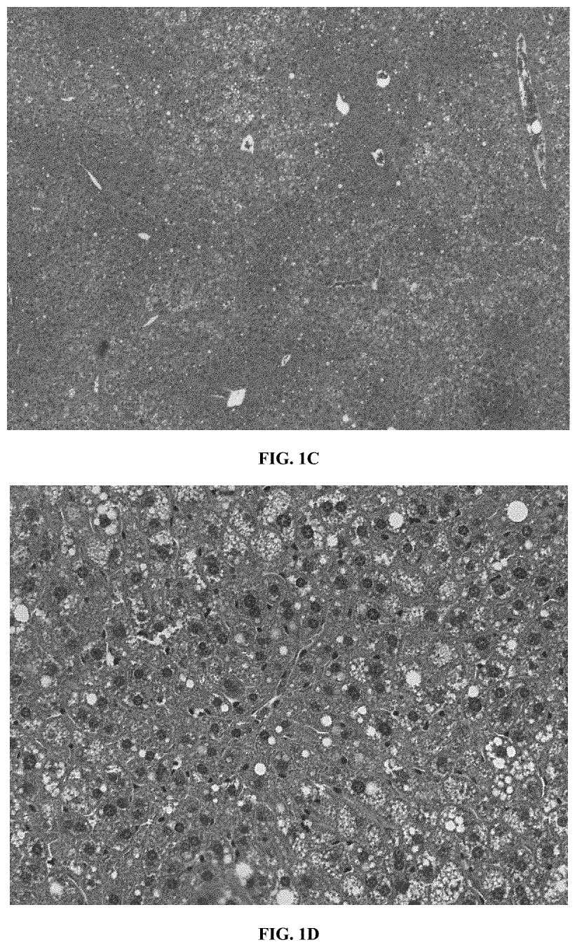

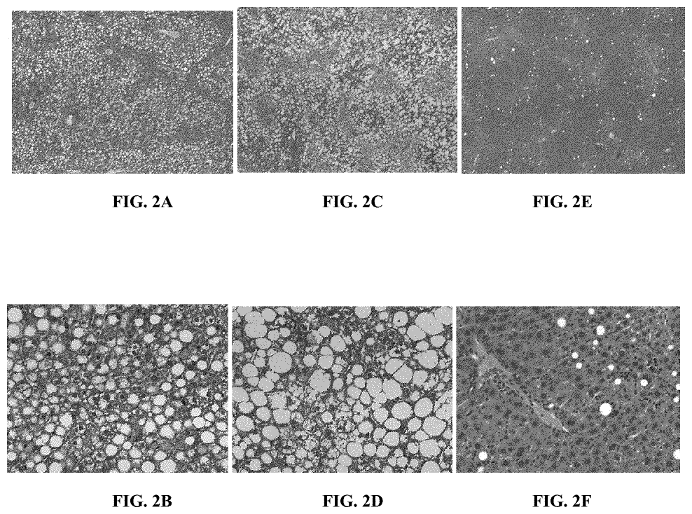

FIGS. 1A-1D show that with increasing age mild steatosis develops even on regular chow. On a HFD, steatosis had developed by 26 weeks, but was significantly reduced after 6 weeks after treatment with the oligonucleotide of SEQ ID NO: 1 (FIGS. 2A-2F).

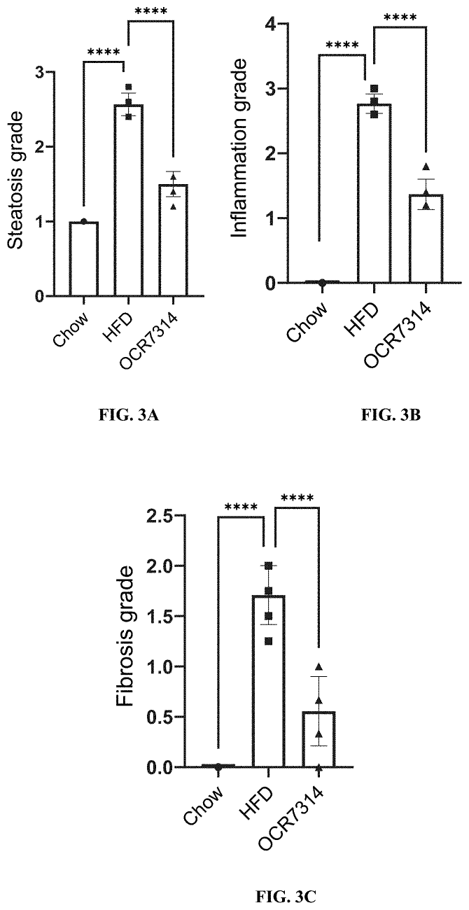

FIGS. 3A-3C show that for three separate assessments of liver tissue, including steatosis grade, inflammation grade, and fibrosis grade, there was a surprising effect observed after the administration of the oligonucleotide of SEQ ID NO: 1 in reducing all three variables.

### Enumerated Embodiments

The following enumerated embodiments are provided, the numbering of which is not to be construed as designating levels of importance:

Embodiment 1 provides a method of treating or ameliorating fibrosis, an inflammatory disorder, or a combination thereof, the method comprising administering a therapeutically effective amount of a composition comprising an oligonucleotide agent comprising the nucleotide sequence of SEQ ID NO: 1 to an individual having fibrosis, an inflammatory disorder, or a combination thereof.

Embodiment 2 provides the method of embodiment 1, wherein treating or ameliorating the inflammatory disorder prevents the inflammatory disorder from progressing to fibrosis.

Embodiment 3 provides the method of any one of embodiments 1-2, wherein the method substantially reduces the cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual, wherein at least a portion of the cellular or plasma levels of the at least one pro-inflammatory cytokine results from activation of Toll-like receptor 9 (TLR9).

Embodiment 4 provides the method of any one of embodiments 1-3, wherein the method substantially reduces cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual, wherein at least a portion of the cellular or plasma levels of the at least one pro-inflammatory cytokine result from activation of TLR9 by mtDNA.

Embodiment 5 provides the method of any one of embodiments 1-4, wherein the fibrosis comprises fibrosis of lung tissue, liver tissue, heart tissue, skin, lymphatic tissue, pancreatic tissue, intestinal tissue, kidney tissue, bone marrow, and/or connective tissue.

Embodiment 6 provides the method of any one of embodiments 1-5, wherein the inflammatory disorder comprises non-alcoholic steatohepatitis (NASH), alcoholic hepatitis, pancreatitis, uveitis, encephalitis, nephritis, encephalitis, bronchitis, pneumonitis, pleuritis, COPD, and/or crush injury.

Embodiment 7 provides the method of any one of embodiments 1-6, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

Embodiment 8 provides the method of any one of embodiments 1-7, wherein the composition is administered to the individual by subcutaneous administration.

Embodiment 9 provides the method of any one of embodiments 1-8, wherein the therapeutically effective amount comprises about 0.01 mg/kg to about 5 mg/kg of the agent.

Embodiment 10 provides the method of any one of embodiments 1-9, wherein the therapeutically effective amount comprises about 0.05 mg/kg to about 2 mg/kg of the agent.

Embodiment 11 provides the method of any one of embodiments 1-10, wherein the agent is administered at a dose of 0.01 mg/kg/week to about 5 mg/kg/week to the individual for about 1 week to about 12 weeks.

Embodiment 12 provides the method of any one of embodiments 1-11, wherein the agent is administered at a dose of 0.01 mg/kg/week to about 5 mg/kg/week to the individual for a first period at a first frequency and for a second period at a second frequency, wherein the second frequency is lower than the first frequency.

Embodiment 13 provides the method of any one of embodiments 1-12, wherein the first period is about 2 weeks to about 6 weeks, the first frequency is weekly, the second period is about 2 weeks to about 10 weeks; and the second frequency is about every 2 weeks to about every 6 weeks.

Embodiment 14 provides the method of any one of embodiments 1-13, wherein the composition comprises at least one pharmaceutically acceptable excipient.

Embodiment 15 provides the method of any one of embodiments 1-14, wherein the composition comprises at least one additional therapeutic agent.

Embodiment 16 provides the method of any of embodiments 1-15, wherein the oligonucleotide agent consists of the nucleotide sequence of SEQ ID NO: 1.

Embodiment 17 provides a method of treating or ameliorating fibrosis, an inflammatory disorder, or a combination thereof, comprising administering a therapeutically effective amount of a composition comprising an oligonucleotide agent comprising the nucleotide sequence of SEQ ID NO: 1 to an individual having fibrosis, an inflammatory disorder, or a combination thereof; wherein the therapeutically effective amount comprises about 0.05 mg/kg to about 2 mg/kg of the agent; wherein the method substantially reduces cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual; and wherein at least a portion of the cellular or plasma levels of the at least one pro-inflammatory cytokine result from activation of TLR9.

Embodiment 18 provides the method of embodiment 17, wherein the oligonucleotide agent consists of the nucleotide sequence of SEQ ID NO: 1.

Embodiment 19 provides a kit, the kit comprising a composition comprising an oligonucleotide agent comprising the nucleotide sequence of SEQ ID NO: 1, an applicator, and instructional material for use thereof, wherein the instructional material comprises instructions for treating or ameliorating fibrosis, an inflammatory disorder, or a combination thereof, in an individual.

Embodiment 20 provides the kit of embodiment 19, wherein the instructional material comprises instructions for administering 0.01 mg/kg/week to about 5 mg/kg/week of the agent to the individual for about 1 week to about 12 weeks.

Embodiment 21 provides the kit of any one of embodiments 19-20, wherein the instructional material comprises instructions for administering 0.01 mg/kg/week to about 5 mg/kg/week of the agent to the individual for a first period at a first frequency and for a second period at a second frequency, wherein the second frequency is lower than the first frequency.

Embodiment 22 provides the method of any one of embodiments 19-21, wherein the instructional material comprises instructions for treating or ameliorating the inflammatory disorder to prevent the inflammatory disorder from progressing to fibrosis, or to induce regression of fibrosis.

Embodiment 23 provides the kit of any one of embodiments 19-22, wherein the fibrosis comprises fibrosis of lung tissue, liver tissue, heart tissue, skin, lymphatic tissue, pancreatic tissue, intestinal tissue, kidney tissue, bone marrow, and/or connective tissue.

Embodiment 24 provides the kit of any one of embodiments 19-23, wherein the inflammatory disorder comprises non-alcoholic steatohepatitis (NASH), alcoholic hepatitis, pancreatitis, uveitis, encephalitis, bronchitis, pneumonitis, pleuritic, nephritis, COPD, and/or crush injury.

Embodiment 25 provides the kit of any one of embodiments 19-24, wherein the applicator is suitable for subcutaneous administration of the composition.

Embodiment 26 provides the kit of any one of embodiments 19-25, wherein the composition comprises at least one additional therapeutic agent.

Embodiment 27 provides the kit of any one of embodiments 19-26, wherein the composition comprises at least one pharmaceutically acceptable excipient.

Embodiment 28 provides the kit of any of embodiments 19-27, wherein the oligonucleotide agent consists of the nucleotide sequence of SEQ ID NO: 1.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A method of treating or ameliorating fibrosis, an inflammatory disorder, or any combinations thereof, the method comprising:
administering a therapeutically effective amount of a composition comprising an oligonucleotide agent comprising the nucleotide sequence of SEQ ID NO: 1 to an individual having fibrosis, an inflammatory disorder, or a combination thereof.

2. The method of claim 1, wherein treating or ameliorating the inflammatory disorder prevents or hampers the inflammatory disorder from progressing to fibrosis.

3. The method of claim 1, wherein the method substantially reduces the cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual, wherein at least a portion of the cellular or plasma levels of the at least one pro-inflammatory cytokine results from activation of Toll-like receptor 9 (TLR9).

4. The method of claim 1, wherein the method substantially reduces cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual, wherein at least a portion of the cellular or plasma levels of the at least one pro-inflammatory cytokine result from activation of TLR9 by mtDNA.

5. The method of claim 1, wherein the fibrosis comprises fibrosis of lung tissue, liver tissue, heart tissue, skin, lymphatic tissue, pancreatic tissue, intestinal tissue, kidney tissue, bone marrow, or connective tissue.

6. The method of claim 1, wherein the inflammatory disorder comprises non-alcoholic steatohepatitis (NASH), alcoholic hepatitis, pancreatitis, uveitis, encephalitis, nephritis, bronchitis, pneumonitis, pleuritis, chronic pulmonary obstructive disease (COPD), or crush injury.

7. The method of claim 1, wherein the composition is administered to the individual by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous administration.

8. The method of claim 7, wherein the composition is administered to the individual by subcutaneous administration.

9. The method of claim 1, wherein the therapeutically effective amount comprises about 0.01 mg/kg to about 5 mg/kg of the agent.

10. The method of claim 6, wherein the therapeutically effective amount comprises about 0.05 mg/kg to about 2 mg/kg of the agent.

11. The method of claim 1, wherein the agent is administered at a dose of 0.01 mg/kg/week to about 5 mg/kg/week to the individual for a first period at a first frequency and for a second period at a second frequency, wherein the second frequency is lower than the first frequency.

12. The method of claim 11, wherein
the first period is about 2 weeks to about 6 weeks;
the first frequency is weekly;
the second period is about 2 weeks to about 10 weeks; and
the second frequency is about every 2 weeks to about every 6 weeks.

13. The method of claim 1, wherein the composition comprises at least one pharmaceutically acceptable excipient.

14. A method of treating or ameliorating fibrosis, an inflammatory disorder, or a combination thereof, the method comprising:
administering a therapeutically effective amount of a composition comprising an oligonucleotide agent comprising the nucleotide sequence of SEQ ID NO: 1 to an individual having fibrosis, an inflammatory disorder, or any combinations thereof;
wherein the therapeutically effective amount comprises about 0.05 mg/kg to about 2 mg/kg of the agent;
wherein the method substantially reduces cellular or plasma levels of at least one pro-inflammatory cytokine chosen from IL-1β, IL-6, IL-8, IL-18, TNFα, IL-4, IL-5, IL-13, IL-17, type 1 interferon, TGF-β, or combinations thereof, in the individual; and
wherein at least a portion of the cellular or plasma levels of the at least one pro-inflammatory cytokine result from activation of TLR9.

15. A kit comprising a composition comprising an oligonucleotide agent comprising the nucleotide sequence of SEQ ID NO: 1, an applicator, and instructional material for use thereof, wherein the instructional material comprises instructions for treating or ameliorating fibrosis, an inflammatory disorder, or a combination thereof, in an individual.

16. The kit of claim 15, wherein the instructional material comprises instructions for administering 0.01 mg/kg/week to about 5 mg/kg/week of the agent to the individual for about 1 week to about 12 weeks.

17. The kit of claim 15, wherein the instructional material comprises instructions for administering 0.01 mg/kg/week to about 5 mg/kg/week of the agent to the individual for a first period at a first frequency and for a second period at a second frequency, wherein the second frequency is lower than the first frequency.

18. The kit of claim 15, wherein the instructional material comprises instructions for treating or ameliorating the inflammatory disorder to prevent or hamper the inflammatory disorder from progressing to fibrosis.

19. The kit of claim 15, wherein the fibrosis comprises fibrosis of lung tissue, liver tissue, heart tissue, skin, lymphatic tissue, pancreatic tissue, intestinal tissue, kidney tissue, bone marrow, or connective tissue.

20. The kit of claim 15, wherein the inflammatory disorder comprises non-alcoholic steatohepatitis (NASH), alcoholic hepatitis, pancreatitis, uveitis, encephalitis, nephritis, bronchitis, pneumonitis, pleuritis, COPD, or crush injury.


SEQUENCE LISTING (2 sequences)

SEQ ID NO: 1 - DNA, 18 bp, Artificial Sequence
  Synthetic sequence
  misc_feature (12)..(12): n is deoxyinosine
tgctcctggagnggttgt

SEQ ID NO: 2 - DNA, 18 bp, Artificial Sequence
  Synthetic sequence
tgctcctggaggggttgt